SEQUENCE LISTING (6 sequences)

SEQ ID NO: 1 - protein, 204 aa, Mus sp.
MDPRTYIEYLNAVTESEKALQSNHFELTLRTEATQGWLVLWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 2 - protein, 212 aa, Mus sp.
MDPRTYIEYLNAVTESELTNEIPAEKALQSNHFELTLRTEATQGWLVLWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 3 - protein, 167 aa, Mus sp.
MDPWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 4 - DNA, 615 bp, Mus sp.
atggatcctcggacctacatcgagtacctcaatgctgtgactgagagtgagaaggcgctgcagagcaaccactttgagctgaccttacgcactgaggccacgcagggctggctcgtcctgtggattggcaaggctgcagaacgtgcagattacatggccctggccatcgtggacgggcacctgcaactgagctatgacctagcttcccagccagttgtcctgcgctccactgtgaaggtcaacaccaaccgctggcttcgagtcagggctcacagggagcacagggagggttcccttcaagtcggcaatgaagcccctgtgacttcctcttccccactgggtgccacgcaactggacacagatggagccctgtggcttggaggcctacagaagcttcctgtgggccagcctctccccaaggcctatggcacgggttttgtgggctgtctgcggaacgtagtgggccatcgccagctgcatctgctggaggacgctgtcaccaaaccagagctaagaccctgccccactctgaattcgaagcttgggcccgaacaaaaactcatctcagaagaggatctgaatagcgccgtcgaccatcatcatcatcatcattga

SEQ ID NO: 5 - DNA, 639 bp, Mus sp.
atggatcctcggacctacatcgagtacctcaatgctgtgactgagagcgagctgaccaatgagatcccagctgagaaggcgctgcagagcaaccactttgagctgaccttacgcactgaggccacgcagggctggctcgtcctgtggattggcaaggctgcagaacgtgcagattacatggccctggccatcgtggacgggcacctgcaactgagctatgacctagcttcccagccagttgtcctgcgctccactgtgaaggtcaacaccaaccgctggcttcgagtcagggctcacagggagcacagggagggttcccttcaagtcggcaatgaagcccctgtgacttcctcttccccactgggtgccacgcaactggacacagatggagccctgtggcttggaggcctacagaagcttcctgtgggccagcctctccccaaggcctatggcacgggttttgtgggctgtctgcggaacgtagtgggccatcgccagctgcatctgctggaggacgctgtcaccaaaccagagctaagaccctgccccactctgaattcgaagcttgggcccgaacaaaaactcatctcagaagaggatctgaatagcgccgtcgaccatcatcatcatcatcattga

SEQ ID NO: 6 - DNA, 504 bp, Mus sp.
atggatccgtggattggcaaggctgcagaacgtgcagattacatggccctggccatcgtggacgggcacctgcaactgagctatgacctagcttcccagccagttgtcctgcgctccactgtgaaggtcaacaccaaccgctggcttcgagtcagggctcacagggagcacagggagggttcccttcaagtcggcaatgaagcccctgtgacttcctcttccccactgggtgccacgcaactggacacagatggagccctgtggcttggaggcctacagaagcttcctgtgggccagcctctccccaaggcctatggcacgggttttgtgggctgtctgcggaacgtagtgggccatcgccagctgcatctgctggaggacgctgtcaccaaaccagagctaagaccctgccccactctgaattcgaagcttgggcccgaacaaaaactcatctcagaagaggatctgaatagcgccgtcgaccatcatcatcatcatcattga

(19) United States
(12) Patent Application Publication
(10) Pub. No.: US 2005/0159354 A1
(43) Pub. Date: Jul. 21, 2005

(54) Reagents and methods for treating epilepsy, traumatic injury and other pathologies of the brain

(76) Inventors: Martin Smith, Irvine, CA (US); Lutz Hilgenberg, San Juan Capistrano, CA (US); Cameron Hoover, Tempe, AZ (US); Diane O'Dowd, Irvine, CA (US)

Correspondence Address: CHRISTIE, PARKER & HALE, LLP, PO BOX 7068, PASADENA, CA, 91109-7068, US

(21) Appl. No.: 11/011,978

(22) Filed: Dec. 13, 2004

Related U.S. Application Data
(60) Provisional application No. 60/529,292, filed on Dec. 12, 2003.

(51) Int. Cl. A61K 38/17

(52) U.S. Cl. 514/12

(57) ABSTRACT

The invention relates to fragments of a mammalian nervous system protein, agrin, and to their use as a therapeutic agent in controlling neural activity associated with epilepsy, traumatic injury and other pathologies of the brain.

### CROSS-REFERENCE TO RELATED APPLICATION(S)

This application claims the benefit of U.S. Provisional Application Ser. No. 60/529,292, filed Dec. 12, 2003, the contents of which is hereby incorporated by reference in its entirety.

### STATEMENT REGARDING FEDERALLY SPONSORED RESEARCH OR DEVELOPMENT

This invention was made with Government support under Grant No. NS33213, awarded by the National Institutes of Health. The Government has certain rights in this invention.

### BACKGROUND OF THE INVENTION

In neuromuscular synaptogenesis, motor neurons secrete the extracellular matrix protein agrin to aggregate acetylcholine receptors (AChR) on the muscle fiber membrane. The details of agrin signaling and AChR clustering are largely a mystery, but muscle-specific kinase (MuSK) has been identified as the agrin receptor that initiates this process. MuSK alone does not form a functional agrin receptor; rather, it forms a heterodimeric complex with an as yet to be identified myotube-associated specificity component (MASC) to transduce its signal. Agrin has also been shown to bind a number of other cell surface components (i.e. laminin, integrin, tenascin, α-dystroglycan, etc.) and it likely has other functions within the peripheral nervous system (PNS), but binding MuSK-MASC and stewarding synaptogenesis at the neuromuscular junction appears to be its most important role.

Determining agrin's interaction with MUSK (and other effector molecules) was facilitated by detailed structural analysis of agrin. Agrin is a ˜400-kD heparan sulfate proteoglycan assembled on an ˜200-kD polypeptide backbone. Conceptually, it can be divided into two parts: nine domains homologous to follistatin and one laminin B-like domain in the N-terminal half of agrin, and four EGF-like domains with three laminin AG-like domains in the C-terminal portion. The N-terminus's tertiary structure is globular, and even though consensus sequences for glycosylation exist throughout the protein, the attachment sites for all heparan sulfate glycosaminoglycan side chains lie in the N-terminal half of the protein. The C-terminal portion connects to the N-terminal half via a central rod and has three globular domains important for receptor binding.

AChR clustering activity resides in the C-terminal half of agrin and further structural analysis of the protein revealed a number agrin isoforms with meaningful exon variations near the signaling domain. These isoforms are expressed differentially in the PNS and CNS and depend upon cell type and developmental stage. Two transcriptional start sites give rise to long and short agrin isoforms and correspond to secreted and membrane bound permutations of the molecule, respectively. Alternative splicing also occurs at three sites within agrin, but the site closest to the C-terminus (the z site) is most important with respect to in vivo agrin function. Analysis of agrin constructs with different exon configurations at the z site not only demonstrated that AChR clustering resides in the third laminin AG-like domain, but also showed AChR clustering is exquisitely sensitive to z+ splice variants. However, CNS cell populations respond to agrin irrespective of exon configuration at the z site, suggesting novel functions and binding partners for agrin in the CNS.

Since MuSK-MASC binding requires an appropriate z+agrin isoform and MuSK is not expressed in the mammalian brain, it is highly probable that agrin mediates its effects via a unique agrin receptor present only in the CNS. Accumulating evidence also suggests that agrin's role in the CNS is broader and more far-reaching than its known function in the PNS. For example, agrin alters rates of axonal and dendritic elongation and inhibits differentiation of presynaptic terminals. Functional agrin also plays a role in permeate ion homeostasis.

A mechanism that links agrin to the distribution of Na⁺ channels has been proposed and agrin deficient neurons demonstrate altered responses to transient changes in cytoplasmic Ca²⁺.

Agrin's involvement in so many fundamental brain processes suggests the potential therapeutic utility of modulating agrin function. Neurofibrillary tangles, senile plaques and amyloid angiopathy characterize the neuropathology of Alzheimer's Disease. Since agrin binds β-amyloid, is a major component of both tangles and plaques, and accelerates fibril formation, its importance to Alzheimer's disease is clear. Agrin's role as a regulator of neuron growth and synaptic plasticity also posits it as a candidate for involvement in traumatic brain injury (TBI) and epilepsy. Axon and dendrite elongation is at least partially regulated by agrin and presents an attractive therapeutic strategy for rescuing neuronal function following TBI. In cultured neurons, agrin influences synaptic efficacy and neural sensitivity to excitatory neurotransmitters. Moreover, heterozygous agrin-deficient mice demonstrate increased resistance to kainic acid-induced seizures. These findings suggest that blockade or control of agrin function following seizures may have therapeutic value in controlling seizure-induced brain injury and/or dampening the kindling phenomena observed seizure disorders. For a review of the role of agrin in the central nervous system, see Smith, M. A. and Hilgenberg, L. G. W. (2002) NeuroReport 13: 1485-1495, herein incorporated by reference in its entirety.

### SUMMARY OF THE INVENTION

The present inventor has shown agrin plays a role in regulating the response of the central nervous system neurons to excitatory neurotransmitters, such as glutamate and nicotine. The present inventor has identified small polypeptide fragments of agrin that (a) are responsible for its biological activity and (b) act as antagonists for the native protein (˜15 kD C-terminal fragments) or which rescue agrin-deficient phenotypes (˜20 kD C-terminal fragments). These polypeptides, or synthetic reagents based on them, may have therapeutic value in situations such as epilepsy or traumatic injury where potentiation or inhibition of agrin's function specifically or general control of the level of neuronal activity may be of benefit. See Hoover, C. L., Hilgenberg, L. G. W. and Smith, M. A., (2003) J. Cell Biol. 161: 923-932, attached hereto and incorporated by reference in its entirety. Because of the specificity of these polypeptides, they are likely to have higher potency and decreased side effects than conventional drugs when used in the therapeutic treatment of epilepsy, traumatic injury to the central nervous system and other disorders of the central nervous system.

Thus, one embodiment of the present invention is directed to a method for controlling seizures in patients diagnosed with epilepsy, comprising administering to an individual diagnosed with epilepsy a therapeutically effective amount of a polypeptide comprising an approximately 15-kD C-terminal agrin fragment.

Another embodiment of the present invention is directed to method for treating traumatic injury to the central nervous system, comprising administering to an individual diagnosed with traumatic injury a therapeutically effective amount of a polypeptide comprising an approximately 15-kD C-terminal agrin fragment.

Still another embodiment of the present invention is directed to method for rescuing an agrin deficient phenotype in an individual in need thereof, comprising administering to the individual a therapeutically effective amount of a polypeptide comprising an approximately 20-kD C-terminal agrin fragment.

Another embodiment of the present invention is directed to method of manufacturing a medicament for use in treating seizures in a mammal, the method comprising:
(a) providing a composition in dosage form, which comprises a synthetic polypeptide comprising an approximately 15-kD C-terminal agrin fragment;
(b) packaging the composition; and
(c) providing the package with a label instructing a user to administer the composition as a medicament for use in treating seizures in a mammal.

Another embodiment of the present invention is directed to method of manufacturing a medicament for use in rescuing an agrin-deficient phenotype in a mammal, the method comprising:
(a) providing a composition in dosage form, which comprises a synthetic polypeptide comprising an approximately 20-kD C-terminal agrin fragment;
(b) packaging the composition; and
(c) providing the package with a label instructing a user to administer the composition as a medicament for use in rescuing an agrin-deficient phenotype in a mammal.

Another embodiment of the present invention is directed to a purified polypeptide, the amino acid sequence of which consists of SEQ ID NO. 1.

Another embodiment of the present invention is directed to a purified polypeptide, the amino acid sequence of which consists of SEQ ID NO. 2.

Another embodiment of the present invention is directed to a purified polypeptide, the amino acid sequence of which consists of SEQ ID NO. 3.

Another embodiment of the present invention is directed to a purified polypeptide, the amino acid sequence of which comprises a sequence at least 70% identical to SEQ ID NO. 1.

Another embodiment of the present invention is directed to a purified polypeptide, the amino acid sequence of which comprises a sequence at least 70% identical to SEQ ID NO. 2.

Another embodiment of the present invention is directed to a purified polypeptide, the amino acid sequence of which comprises a sequence at least 70% identical to SEQ ID NO. 3.

Another embodiment of the present invention is directed to a purified polypeptide, the amino acid sequence of which comprises SEQ ID NO. 1, or SEQ ID NO. 1 with at least one conservative amino acid substitution.

Another embodiment of the present invention is directed to a purified polypeptide, the amino acid sequence of which comprises SEQ ID NO. 2, or SEQ ID NO. 2 with at least one conservative amino acid substitution.

Another embodiment of the present invention is directed to a purified polypeptide, the amino acid sequence of which comprises SEQ ID NO. 3, or SEQ ID NO. 3 with at least one conservative amino acid substitution.

### BRIEF DESCRIPTION OF THE FIGURES

FIG. 1 is a schematic diagram showing the structure of agrin and agrin deletion constructs. Alternate transcriptional start sites give rise to short and long NH2-terminal (SN, LN) forms of agrin. Agrin's polypeptide chain is characterized by numerous cysteine-rich repeats similar to follistatin (F), laminin B (LB), EGF (E), and laminin A G domains (G). Two serine/threonine-rich regions (S/T), consensus glycosaminoglycans side-chain attachment sites (lollipops), and sites of alternative splicing (X, Y, Z) are also shown. Horizontal bars indicate location of binding sites for various cell surface and ECM molecules. The minimal region required for agrin's AChR clustering activity is also shown. Agrin deletion constructs C-Ag95_{z0/8} and C-AgΔ20 included an NH₂-terminal signal peptide for expression in mammalian cells and 4 amino acid insert at the y site. All deletion constructs included COOH-terminal myc (m) and polyhistidine (H) epitope tags.

FIG. 2 shows induction of expression of c-fos in cultured cortical neurons by C-Ag95_{z8} and C-Ag95_{z0}. (A) 12d-old cortical cultures were treated for 10 min with either C-Ag95_{z8} or C-Ag95_{z0}, followed by double labeling with antibodies for Fos (fluorescein channel) and either MAP2 or GFAP (rhodamine channel). Cell bodies and nuclei of MAP2-positive neurons were intensely labeled for Fos in cultures treated with either C-Ag95_{z8} or C-Ag95_{z0}. In contrast, only basal levels of Fos expression were observed in GFAP-positive normeuronal cells. Induction of c-fos was agrin specific in that no detectable increase in Fos was apparent in cultures treated with prostate serum antigen control protein. Bar, 20 μm. (B) Cultures were incubated for 10 min in C-Ag95_{z8} (open circles, broken line) or C-Ag95_{z0} (filled circles, solid line), and levels of Fos expression were determined by in situ enzyme-linked assay as described herein. Both agrin constructs induced essentially identical concentration dependent and saturable increases in c-fos expression. Data were normalized to maximal level of Fos expression in each experiment. Each point represents the mean of triplicate determinations from three independent experiments±SEM. Curves were fit by single-site nonlinear regression model R²≧0.94. Values from mock-treated sister wells have been subtracted.

FIG. 3 is a graph showing that the 20-kD COOH-terminal region of agrin is necessary and sufficient for induction of c-fos. (A) Cortical neurons were treated for 10 min with C-AgΔ20 alone or in the presence of 50 pM C-Ag95_{z8} or C-Ag95_{z0}. Neither a subsaturating (50 pM) nor supersaturating (50 nM) concentration of C-AgΔ20 induced expression of c-fos, nor were they able to modulate the activity of the larger fragments. (B) In contrast, cortical cultures exhibited a concentration-dependent and saturable increase in response to C-Ag20_{z8} (open circles, broken line) or C-Ag20_{z0} (solid circles, solid line) similar to that seen with the C-Ag95_{z8/0} fragments. Data were normalized to the level of Fos expression induced by 50 pM C-Ag95_{z8} alone in A and maximal level of Fos expression in B. Each data point shows the mean of triplicate determinations from three independent experiments±SEM. Curves were fit by single-site nonlinear regression model R²≧0.97. Background levels of Fos expression from mock-treated sister cultures have been subtracted.

FIG. 4 is a graph showing that the 15-kD COOH-terminal region of agrin is a cell-specific competitive inhibitor of C-Ag95_{z8} induction of c-fos. (A) Cortical cultures were incubated in 50 pM C-Ag95_{z8} in the presence of different concentrations of C-Ag15. C-Ag15 inhibited C-Ag95_{z8} induction of c-fos in a concentration-dependent manner well described by a single-site competition model (R²=0.95) with an IC₅₀ of 64.5 pM; close to a 1:1 agonist:antagonist molar ratio. Data were normalized to percentage of Fos expression induced by 50 pM C-Ag95_{z8} alone, and represent the mean of triplicate determinations from three independent experiments±SEM. Background levels of Fos expression have been subtracted. (B) To test the cell specificity of C-Ag15 inhibition, hippocampal (Hip) or cerebellar (Cer) neurons and chick muscle fibers (Mus) were incubated in 50 pM C-Ag95_{z8} alone (open bars) or in the presence (filled bars) of 1 nM C-Ag15. Levels of Fos expression were expressed as fold change over mocktreated sister cultures such that a value of 1 indicates no change. C-Ag15 completely inhibited C-Ag95_{z8}-induced expression of Fos in hippocampal and cerebellar neurons, but not in muscle. (C) The effect of C-Ag15 on C-Ag95_{z8}-induced AChR aggregation was also tested. Cultured chick myotubes were incubated overnight in 50 pM C-Ag95_{z8} alone (−) or in the presence (+) of 1 nM C-Ag15, and AChR clusters were labeled with rhodamine-conjugated α-bungarotoxin. AChR clusters were counted blind with respect to treatment in five random fields/well and expressed as the ratio of clusters in mock-treated sister cultures. Consistent with the results of the Fos expression analysis, C-Ag15 had no effect on C-Ag95_{z8}-induced AChR clustering. Bars in B and C represent the mean±SEM of triplicate wells from four independent experiments.

FIG. 5 is a graph showing that the 20-kD COOH-terminal region of agrin is the minimal fragment sufficient to rescue an agrin-deficient phenotype. (A) 10-d-old wild-type (filled bars) or agrin-deficient (open bars) cultures were grown for 2 d in media supplemented to 5 nM with the indicated agrin fragment. Cultures were challenged for 5 min with 100 μM glutamate as described previously (Hilgenberg et al., 2002), and the levels of Fos expression were determined. Data were normalized to levels of glutamate-induced Fos expression in mocktreated wild-type cultures. Compared with mock, glutamate responses of homozygous agrin-deficient neurons were rescued to near wildtype levels by either the long C-Ag95_{z8/0} or short C-Ag20_{z8/0} fragments (***, P≦0.0005; **, P≦0.002; *, P≦0.02; paired t test). (B) To test the ability of C-Ag15 to antagonize agrin action, 10-d-old agrin-deficient neurons were maintained for 2 d in the presence of 5 nM C-Ag95_{z0} alone or in combination with 10 nM C-Ag15, and neuronal responses to glutamate were determined as above. Treatment with C-Ag15 significantly (*, P≦0.004; paired t test) inhibited rescue of the agrin-deficient response to glutamate by C-Ag95_{z0}.

FIG. 6 is a microphotograph showing agrin receptor expression in nerve cell membranes. Live cortical neurons were incubated in C-Ag20_{z8}, C-Ag20_{z0}, or C-Ag15, either alone or in the presence of mock-conditioned medium or a 500-fold molar excess of the active (Rat C-Ag_{4,8}) or inactive (Rat C-Ag_{0,0}) isoform of rat agrin. Immunostaining with an anti-polyhistidine antibody reveals binding of the short agrin fragments to receptors distributed in numerous small clusters on neuron cell bodies and neurites; patches of agrin receptors outside the focal plane contribute to the diffuse staining evident in some neuron cell bodies. Consistent with a single class of agrin receptors, each fragment shows a similar pattern of binding that can be blocked by either isoform of rat agrin. The ability of rC-Ag to block the short agrin fragments is also strong evidence that binding is specific. In addition, no labeling was observed when the short agrin fragments were omitted or replaced by β-galactosidase (β-Gal) as a control for vector-specific sequences. Bar, 20 μm.

FIG. 7 is a microphotograph showing agrin and agrin receptors colocalized at synaptic contacts. The subcellular distribution of endogenous agrin and agrin receptors on cultured cortical neurons was determined by labeling with either an anti-agrin serum, RαAg-1, or short agrin fragment, followed by fixation and incubation with an antibody to synaptophysin to identify nerve terminals by labeling synaptic vesicles. Both agrin and the agrin receptor were present at virtually all synaptophysin-positive nerve terminals (arrowheads), evidence that agrin and its receptor are colocalized at synaptic sites. Nerve terminal staining was specific, and was not observed in control cultures labeled with an agrin receptor probe in the absence of the anti-synaptophysin antibody. Bar, 20 μm.

FIG. 8. Agrin blocks spontaneous action potentials in cultured cortical neurons. (A) Spontaneous action potentials were reversibly blocked by application of a saturating concentration of rat C-Ag95_{4.8}. Similar results were obtained in 5/5 neurons from which stable recordings were obtained. (B) In 3/3 neurons examined, no change in spike frequency was observed following treatment with conditioned medium harvested from sham transfected cas cells but action potentials were blocked by subsequent treatment with C-Ag95_{4,8}— (C) Complete but reversible blockade of action potentials was also observed in 7/7 neurons treated with 10 pM C-Ag20₈. Note the slow wave activity initially present in this record was increased following C-Ag20₈ treatment but returned to normal levels upon washout. All extracellular recordings were obtained using a cell attached patch configuration.

FIG. 9. Agrin increases activity at inhibitory synapses. (A) Treatment with C-Ag20₈ reversibly increases the frequency and amplitude of sIPSCs. Records were obtained from a single neuron. Neuron was returned to normal saline and exhibited baseline levels of sIPCS between C-Ag20₈ trials. (B) Consistent with our previous findings, synaptic activity does not change in the presence of C-Ag15. However, C-Ag15 is an effective antagonist for C-Ag20₈. This cell could respond to agrin, as shown in the bottom record showing the response to C-Ag20₈ after C-Ag15 had been washed out. (C) C-Ag20₈ induced changes in sIPSC frequency are concentration dependent. Number of neurons in each category is given. (D) A cumulative probability plot of IPSC amplitudes recorded from a single neuron. Compared to saline (solid line) there is an increase in the fraction of large IPSCs in presence of C-Ag20₈ (50 pM; broken line).

### DESCRIPTION OF PREFERRED EMBODIMENTS

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Definitions. The following terms are used herein:

“Individual” means any living organism, including humans and other mammals, which produce agrin.

“Native agrin” or “agrin” is an ˜400-kD heparan sulfate proteoglycan assembled on an ˜200-kD polypeptide backbone characterized by multiple cysteine-rich domains.

“C-Ag20” or “C-Ag20_{z0/z8}” refers to the 20-kD COOH-terminal fragment of agrin containing the alternatively spliced z site.

“C-Ag20_{z0}” refers to the C-Ag20 isoform having the z0 splice variant. The amino acid sequence of C-Ag20_{z0} is shown as SEQ. ID NO. 1; the nucleic acid sequence encoding C-Ag20_{z0} is shown as SEQ. ID NO. 4.

“C-Ag20_{z8}” refers to the C-Ag20 isoform having the z8 splice variant. The amino acid sequence of C-Ag20_{z8} is shown as SEQ. ID NO. 2; the nucleic acid sequence encoding C-Ag20_{z8} is shown as SEQ. ID NO. 5.

“C-Ag15” refers to the 15-kD COOH-terminal fragment of agrin created by deleting 37 amino acids from the NH₂ terminus of C-Ag20. The amino acid sequence of C-Ag15 is shown as SEQ. ID NO. 3; the nucleic acid sequence encoding C-Ag15 is shown as SEQ. ID NO. 6.

“Homologs” refers to polypeptides in which one or more amino acids have been replaced by different amino acids, such that the resulting polypeptide is at least 75% homologous, and preferably at least 85% homologous, to the basic sequence as, for example, the sequence of agrin, C-Ag20 or C-Ag15, and wherein the variant polypeptide retains the activity of the basic protein, for example, agrin, C-Ag20 or C-Ag15. Homology is defined as the percentage number of amino acids that are identical or constitute conservative substitutions. Conservative substitutions of amino acids are well known in the art. Representative examples are set forth in Table 1.

TABLE 1

| Original Residue | Conservative Substitution (s) |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Homologs of polypeptides may be generated by conventional techniques, including either random or site-directed mutagenesis of DNA encoding the basic polypeptide. The resultant DNA fragments are then cloned into suitable expression hosts such as E. coli or yeast using conventional technology and clones that retain the desired activity are detected. The term “homolog” also includes naturally occurring allelic variants.

“Derivative” refers to a polypeptide that has been derived from the basic sequence by modification, for example by conjugation or complexing with other chemical moieties or by post-translational modification techniques as would be understood in the art. Such derivatives include amino acid deletions and/or additions to polypeptides or variants thereof wherein said derivatives retain activity of the basic protein, for example, agrin, C-Ag20 or C-Ag15. Other derivatives contemplated by the invention include, but are not limited to, modification to side chains, incorporation of unnatural amino acids and/or their derivatives during peptide, polypeptide or protein synthesis and the use of crosslinking agents.

In addition to polypeptides consisting only of naturally-occurring amino acids, the present invention includes peptidomimetics. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15: 29; Veber and Freidinger (1985) TINS p. 392; and Evans et al. (1987) J. Med. Chem 30: 1229, which are incorporated herein by reference) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH₂NH—, —CH₂S—, —CH₂CH₂—, CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14: 177-185 (—CH₂NH—, —CH₂CH₂—); Spatola, A. F. et al., Life Sci (1986) 38: 1243-1249 (—CH₂S—); Hann, M. M., J Chem Soc Perkin Trans I (1982) 307-314 (—CH═CH—, cis and trans); Almquist, R. G. et al., J Med Chem (1980) 23: 1392-1398 (—COCH₂—); Jennings-White, C. et al., Tetrahedron Lett (1982) 23: 2533 (—COCH₂—); Szelke, M. et al., European Appln. EP 45665 (1982) CA: 97: 39405 (1982) (—CH(OH)CH₂—); Holladay, M. W. et al., Tetrahedron Lett (1983) 24: 4401-4404 (—C(OH)CH₂—); and Hruby, V. J., Life Sci (1982) 31: 189-199 (—CH₂S—); each of which is incorporated herein by reference. A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) (e.g., receptor molecules) to which the peptidomimetic binds to produce the therapeutic effect. Derivitization (e.g., labeling) of peptidomimetics should not substantially interfere with the desired biological or pharmacological activity of the peptidomimetic.

“Therapeutic composition” is defined as comprising C-Ag20 or C-Ag15 and a pharmaceutically acceptable carrier.

A “pharmaceutically acceptable carrier” is a solid or liquid filler, diluent or encapsulating substance that may be safely used in systemic administration. Depending upon the particular route of administration, a variety of pharmaceutically acceptable carriers, well known in the art can be used. These carriers include, but are not limited to, sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, 16th Edition, Mack, (1980)).

Where a particular polypeptide or nucleic acid molecule is said to have a specific percent identity or conservation to a reference polypeptide or nucleic acid molecule, the percent identity or conservation can be determined by the algorithm of Myers and Miller, CABIOS (1989), which is embodied in the ALIGN program (version 2.0), or its equivalent, using a gap length penalty of 12 and a gap penalty of 4 where such parameters are required. All other parameters are set to their default positions. Access to ALIGN is readily available. See, e.g., http://www2.igh.cnrs.fr/bin/align-guess.cgi on the internet.

Comparison of the sequence to the data bases can be performed using BLAST (Altschcul, S. F. et al., J. Mol. Biol. 215: 403-410 (1990)).

Parameters for polypeptide sequence comparison include the following: (1) Algorithm: Needleman and Wunsch, J. Mol. Biol. 48: 443-453 (1970); (2) Comparison matrix: BLOSSUM62 from Hentikoff and Hentikoff, Proc. Natl. Acad. Sci. USA. 89: 10915-10919 (1992); (3) Gap Penalty: 12; and (4) Gap Length Penalty: 4. A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: (1) Algorithm: Needleman and Wunsch, J. Mol. Biol. 48: 443-453 (1970); (2) Comparison matrix: matches=+10, mismatch=0; (3) Gap Penalty: 50; and (4) Gap Length Penalty: 3. Available as: The “gap” program from Genetics Computer Group, Madison Wis. These are the default parameters for nucleic acid comparisons.

Polypeptides of the invention may be prepared by any suitable procedure known to those of skill in the art. Recombinant polypeptides of the invention may be produced by culturing a host cell transformed with an expression vector containing nucleic acid encoding a polypeptide, fragment, homolog or derivative according to the invention. Recombinant protein may be conveniently prepared by a person skilled in the art using standard protocols as, for example, described in Sambrook, et al., MOLECULAR CLONING. A LABORATORY MANUAL (Cold Spring Harbor Press, 1989), incorporated herein by reference, in particular Sections 16 and 17; Ausubel et al., CURRENT PROTOCOLS IN MOLECULAR BIOLOGY (John Wiley & Sons, Inc. 1994-1998), incorporated herein by reference, in particular Chapters 10 and 16; and Coligan et al., CURRENT PROTOCOLS IN PROTEIN SCIENCE (John Wiley & Sons, Inc. 1995-1997) which is incorporated by reference herein, in particular Chapters 1, 5 and 6. Examples of vectors suitable for expression of recombinant protein include but are not limited to pGEX, pET-9d, pTrxFus or baculovirus (available from Invitrogen). A number of other vectors are available for the production of protein from both full length and partial cDNA and genomic clones, producing both fused or non-fused protein products, depending on the vector used. The resulting proteins are frequently immunologically and functionally similar to the corresponding endogenous proteins.

The obtained polypeptide is purified by methods known in the art. The degree of purification varies depending on the use of the polypeptide. For use in eliciting polyclonal antibodies, for example, the degree of purity may not need to be very high. However, as in some cases impurities may cause adverse reactions, purity of 90-95% is typically preferred and in some instances even required. For the preparation of a therapeutic composition, however, the degree of purity must be high, as is known in the art.

The present invention provides for the administration of a therapeutic composition comprising C-Ag20 or C-Ag15, or homologs, derivatives or peptidomimetics thereof, to an individual diagnosed with epilepsy, traumatic injury or other pathologies of the brain. In a preferred embodiment of the present invention, therapeutic compositions comprising C-Ag15, or homologs, derivatives or peptidomimetics thereof, are administered to inhibit agrin function to thereby control seizures associated with epilepsy, traumatic brain injury, and other disorders of the central nervous system in which agrin is shown to affect biological activity. In another preferred embodiment of the present invention, therapeutic compositions comprising C-Ag20, or homologs, derivatives or peptidomimetics thereof, are administered to rescue an agrin-deficient phenotype.

It is understood that the dosage of the therapeutic composition of the present invention administered in vivo or in vitro will be dependent upon the age, sex, health, and weight of the recipient, kind of concurrent treatment, if any, frequency of treatment, and the nature of the pharmaceutical effect desired. The most preferred dosage will be tailored to the individual subject, as is understood and determinable by one skilled in the relevant arts. See, e.g., Berkow et al., eds., The Merck Manual, 16th edition, Merck and Co., Rahway, N.J. (1992); Goodman et al., eds., Goodman and Gilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987); Ebadi, Pharmacology, Little, Brown and Co., Boston (1985); Osol et al., eds., Remington's Pharmaceutical Sciences, 17th edition, Mack Publishing Co., Easton, Pa. (1990); Katzung Basic and Clinical Pharmacology, Appleton and Lange, Norwalk, Conn., (1992), which references are entirely incorporated herein by reference.

The term “therapeutically effective amount” as used herein, means that amount of C-Ag20 or C-Ag15 that elicits the biological or medicinal response in a tissue system, animal or human that is being sought by a researcher, veterinarian, medical doctor or other clinician, which includes alleviation of the symptoms of the disease or disorder being treated.

The total dose required for each treatment can be administered by multiple doses or in a single dose. The diagnostic/pharmaceutical compound or composition can be administered alone or in conjunction with other diagnostics and/or pharmaceuticals directed to the pathology, or directed to other symptoms of the pathology.

The therapeutic composition of the invention may be administered by any of the conventional routes of administration, including oral, rectal, parenteral, sublingual, buccal, intravenous, intra-articular, intramuscular, intra-dermal, subcutaneous, inhalational, intraocular, intraperitoneal, intracerebroventricular, transdermal and the like, or as described in U.S. Pat. No. 5,693,607, the entire contents of which is hereby incorporated by reference. Also, the therapeutic composition of the invention may be in any of several conventional dosage forms, including, but not limited to, tablets, dispersions, suspensions, injections, solutions, capsules, suppositories, aerosols, and transdermal patches.

The invention also includes recombinant DNA vectors containing a gene encoding agrin, or fragments or variants thereof, preferably vectors that target neuronal cells, as, for example, by targeting overexpressed cell surface receptors.

The invention also contemplates polyclonal, monoclonal and humanized antibodies against the aforementioned agrin polypeptides, fragments, homologs and derivatives.

Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991) which is incorporated herein by reference, and Ausubel et al., (1994-1998, supra), in particular Section III of Chapter 11.

Alternatively, monoclonal antibodies may be produced using the standard method as, for example, described in an article by Kohler and Milstein (1975, Nature 256, 495-497) which is herein incorporated by reference.

According to the method of the current invention, large amounts of recombinant agrin, or derivatives, homologs or fragments thereof, are produced by scale up processes in commercial plants which enables production of a corresponding large quantity of antibodies. The antibodies to recombinant expressed protein can also be produced according to the invention using the standard method available for production of the antibodies to native protein.

The antibodies of the invention may be used for affinity chromatography in isolating natural or recombinant agrin polypeptides or fragments thereof. The antibodies can also be used to screen expression libraries for variant polypeptides of agrin. Preferably, the antibodies of the invention can be administered to individuals diagnosed with epilepsy, traumatic injuries and other pathologies of the brain. Preferably, humanized antibodies (XENOMOUSE®, Abgenix, Inc., Fremont, Calif.; Bodey B., et al., Curr. Pharm. Des. 6: 261-76 (2000); Halloran P. F., et al., Clin. Biochem. 31: 353-7 (1998).) are administered as therapeutic agents to treat epilepsy, traumatic injuries and other pathologies of the central nervous system.

Antibodies may administered as described above for therapeutic compositions. Preferably, therapeutic antibodies are administered either subcutaneously or by intravenous injection.

### EXAMPLES

### Materials and Methods

Tissue culture. Mouse cortical cultures were prepared from newborn or 1-d-old ICR strain mice (Harlan) as described previously (Hilgenberg et al., 1999). For the first 24 h after plating, cells were maintained in neural basal medium (NBM) plus B27 supplements (Invitrogen), and in normeuronal cell-conditioned NBM (cNBM) plus B27 thereafter, at 37° C. in humidified 5% CO₂ atmosphere. To further reduce proliferation of nonneuronal cells, cultures on glass coverslips used for histology were treated with 5 μM 5-fluoro-2-deoxyuridine (Sigma-Aldrich) 3-4 d after plating. Hippocampal and cerebellar cultures were prepared in a similar manner. Experiments were performed on 10-14-d-old cultures.

Agrin-deficient neuron cultures were prepared from cortices of embryonic d 18-19 fetuses resulting from matings between mice heterozygous for a mutation in the agrin gene (Gautam et al., 1996). Cultures were prepared and genotyped as described previously (Li et al., 1999), and were maintained as above.

Chick muscle cultures were prepared from pectoral muscles of 10-11-d-old White Leghorn chick embryos as described previously (Hilgenberg et al., 1999). Experiments were performed on 4-6-d-old cultures.

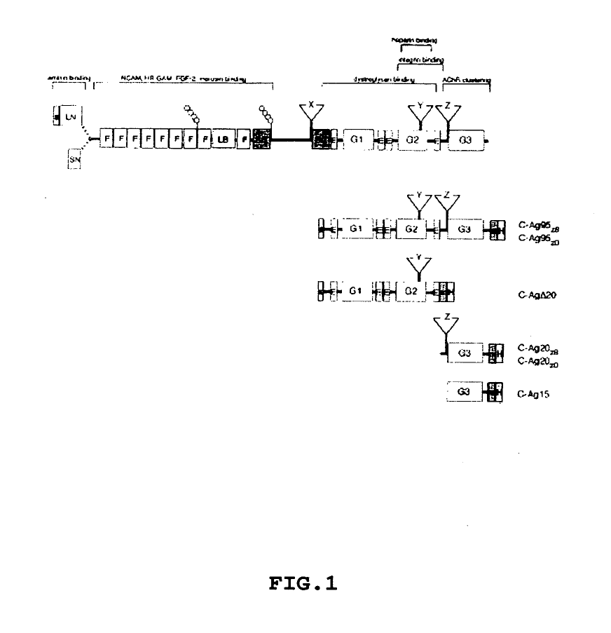

Expression constructs. Parent constructs, C-Ag95_{z0} and C-Ag95_{z8} (FIG. 1), encoding the soluble 95-kD COOH terminus of mouse agrin, were generated from cDNA prepared by RT-PCR of adult mouse cortex RNA using the F95/R95 primer pair (see Table I) subcloned into the pGEM-T (Promega) shuttle vector and transformed into JM109-competent bacteria. Individual ampicillin-resistant colonies were picked, and C-Ag95_{z0} and C-Ag95_{z8}-containing clones were identified by PCR analysis using primers F24/B2 flanking the z site. After double digestion of the BamHI and EcoRV 1 linker sites contained within the F95 and R95 primers, agrin inserts were gel purified and ligated into the pSecTag2B expression vector (Invitrogen) in frame with a COOH terminal myc epitope and 6× polyhistidine tag.

C-AgΔ20 lacking the 20-kD COOH-terminal region of C-Ag_{z0/8} was prepared by PCR amplification of the C-Ag95_{z8} pGEM-T template using F95 and the reverse primer RΔ20 that includes a 3′ EcoRV site. DNA from the PCR reaction was digested with BamHI and EcoRV, gel purified, and ligated into pSecTag2B.

A similar strategy was used to generate C-Ag20_{z0/8} and C-Ag15 constructs by PCR amplification of the appropriate pGEM-T C-Ag_{z0/8} template using either F20 (for C-Ag20_{z0/8}) or F15 (for C-Ag15) in combination with R20. However, for more efficient expression, aliquots of the PCR reaction were ligated into the inducible bacterial expression vector pTrcHis2 (Invitrogen).

TABLE I

| | | | |
|---|---|---|---|
| Construct | Primer | Sequence | Nucleotide |

| | | | |
|---|---|---|---|
| C—Ag_{y4z0/8} | F95 | TAGGATCCACCGCCAGTATTGACCGA | 1104-1121 |
| | R95 | TAGATATCAGAGTGGGGCAGGGTCTT | 6678-6663 |
| C—AgΔ20 | RΔ20 | TAGATATCGTCCGCCCATCAAAGGCC | 4585-4568 |
| C—Ag20_{z0/8} | F20 | AATGGATCCTCGGACCTACATCG | 4581-4593 |
| | R20 | TTCGAATTCAGAGTGGGGCAGGG | 6679-6666 |
| C—Ag15 | F15 | AATGGATCCGTGGATTGGCAAGGC | 5750-5764 |

Expression and quantitation of recombinant agrin. pSecTag2B agrin vector DNA encoding either C-Ag_{z0/8} or C-AgΔ20 was transfected into HEK 293T cells using LipofectAMINE™ (Invitrogen) according to the manufacturer's directions. Controls were either sham transfected with LipofectAMINE™ alone or with control vector encoding prostate-specific antigen (pSecTag2-PSA). Agrin constructs in the pTrcHis2 expression vector were maintained in the JM109 bacteria. The plasmid pTrcHis2-lacZ encoding β-galactosidase was expressed as a control.

Polyhistidine-tagged agrin fragments were purified from conditioned media and bacterial extracts using the Talon™(CLONTECH Laboratories, Inc.) metal affinity resin eluted with 200-500 mM imidazole (Sigma-Aldrich) according to the manufacturer's instructions. The identity of the isolated fragments was confirmed by immunoblot analysis using RαAg-1, a rabbit antiserum raised against a synthetic peptide corresponding to amino acids 1862-1895 conserved in all isoforms of mouse agrin. For some experiments, the elution buffer was removed by dialysis against PBS or 20 mM Tris and 250 mM NaCl, pH 8.0.

The molar concentration of each fragment was determined by comparison to a C-Ag95_{z0} standard prepared as follows: HEK 293T cells were transfected with pSecTag2B-C-Ag_{z0}, and were then transferred to 80% methionine-free DME containing 100 μCi/ml [³⁵S]methionine. C-Ag95_{z0} present in the medium was purified over a Talon™ metal affinity resin column, and the apparent molar concentration was determined by counting aliquots of the column eluate in a scintillation counter (model LS7500; Beckman Coulter). A small correction factor was applied to account for the fraction of counts incorporated into C-Ag95_{z0} (≧90%) versus total counts determined by phosphorimager analysis (Molecular Dynamics, Inc.) of an aliquot of the eluate separated on an 8% SDS-PAGE gel.

The concentration of the other agrin fragments was determined by comparison to a ³⁵S-labeled C-Ag95_{z0} standard in immunoblots probed with a mouse anti-myc antibody (Invitrogen) and ¹²⁵I-labeled anti-mouse second antibody (Amersham Biosciences). The amount of ¹²⁵I bound to both the standard and unknown was determined by phosphorimager analysis and, after correcting for the contribution of the [³⁵S]methionine in the standard, the concentration of the unknown was determined from the standard curve.

Soluble 95-kD COOH-terminal fragments of rat agrin (rC-Ag_{y4z8/y0z0}) were harvested in media conditioned by transiently transfected COS-7 cells (Hilgenberg et al., 1999) and dialyzed against PBS. The concentration of the rC-Ag was estimated by comparison to a mouse agrin standard in the c-fos induction assay and by immunoblot analysis with RαAg-1.

Quantitative analysis of Fos expression. Fos expression in cortical cultures was measured by in situ enzyme-linked immunoassay (Hilgenberg et al., 1999). In brief, 11-14-d-old neuronal cultures were treated for 10 min with agrin or other agent diluted in NBM or PBS, then washed in CNBM and returned to the incubator for 2 h. Cultures were rinsed in PBS, fixed in ice cold 4% PFA, and blocked in PBS containing 0.1% Triton X-100 and 4% BSA (PBSTB) before being incubated in a primary rabbit antibody against Fos (Ab-2; Oncogene Research Products) and secondary goat antibody against mouse conjugated to alkaline phosphatase (Southern Biotechnology Associates, Inc.). The level of Fos expression was determined by monitoring conversion of p-nitrophenyl phosphate to a soluble yellow reaction product at 405 nm.

AChR clustering assay. 4-6-d-old myotubes were treated with agrin overnight followed by incubation with 20 nM rhodamine-conjugated α-bungarotoxin (Molecular Probes, Inc.) in culture medium for 1 h at 37° C. Cells were then fixed in 4% PFA in PBS, washed in PBS, and viewed at 200× under epifluorescent illumination on a microscope (Optiphot-2; Nikon). For each well, the mean number of AChR clusters/field was determined from counts obtained from five random fields. All counts were performed blind with respect to treatment. To facilitate comparison between experiments, the number of AChR clusters/field was normalized to the cluster density of control cultures treated with vehicle alone.

Fos immunohistochemistry. 10-14-d-old cortical neurons, grown on glass coverslips, were treated with agrin, fixed, and blocked as for the Fos in situ enzyme-linked immunoassay. Cells were double labeled overnight at 4° C. with Ab-2 (1:200) together with either a mouse monoclonal antibody against MAP2 (SMI-52; 1:400; Sternberger Monoclonals), or GFAP (G-A-5; 1:1,000; Sigma-Aldrich) in PBSTB to identify neurons or glial cells, respectively. Bound antibodies were visualized by incubation for 2 h at RT in a mixture of fluorescein-conjugated goat anti-rabbit and Texas red-labeled goat anti-mouse secondary antibodies (Vector Laboratories) diluted 1:200 in PBSTB. Coverslips were washed in PBS, mounted in Fluoromount™ (Southern Biotechnology Associates, Inc.), and examined using epifluorescent illumination.

Agrin immunohistochemistry. Live cortical cultures on glass coverslips were incubated for 30 min at 37° C. in R□Ag-1 diluted 1:500 in NBM. Cultures were then washed in NBM, fixed and blocked as described for the Fos in situ enzyme-linked immunoassay, and were then washed and incubated overnight at 4° C. with the anti-synaptophysin antibody, SVP38 (Sigma-Aldrich), diluted 1:400 in PBS containing 4% BSA (PBSB). Primary antibodies were visualized by double labeling with fluorescein-conjugated goat anti-rabbit and Texas red-labeled anti-mouse antibodies as above.

Agrin receptor immunohistochemistry. The distribution of agrin receptors was studied using agrin deletion fragments as affinity probes. Neurons, plated on glass coverslips, were washed briefly (1-2 min) in cold PBS containing 10 mM EDTA followed by a second wash in cold PBS alone before incubation for 15 min at 4° C. with 1 pM recombinant mouse agrin in NBM. In some experiments, labeling with mouse agrin was performed in the presence of various concentrations of rat rC-Ag95y4_{z8} or rC-Agy_{0z0}. Control cultures were treated with vector control protein or an equivalent volume of vehicle in which the recombinant agrin was dissolved. Cells were then washed in cold NBM, fixed for 40 min on ice in 4% PFA in PBS, then washed in PBS and blocked for 1 h in PBSB. Recombinant agrin was detected through the COOH-terminal 6× polyhistidine tag using an anti-his antibody (1:500; Invitrogen). In experiments where cells were double labeled for synaptophysin, the anti-synaptophysin antiserum (1:400) was also added at this step. Incubations were performed overnight at 4° C. in PBSB, followed by labeling with fluoresceinconjugated goat anti-mouse and Texas red-labeled anti-rabbit antibodies as described in the previous paragraphs. In some experiments, the amount of mouse agrin bound was estimated by digital photomicrography using the public domain NIH Image program (http://rsb.info.nih.gov/nih-image/).

### Example 1

### Agrin Signaling in Neurons is Independent of Splicing at the z Site

Initial characterization of the agrin signal transduction pathway in CNS neurons demonstrated its inability to discriminate between “active” z+ and “inactive” z agrin isoforms (Hilgenberg et al., 1999). However, these works used alternatively spliced variants of the 95-kD COOH-terminal region of rat agrin (rC-Ag_{z0/8}), and were limited by the fact that only indirect estimates of agrin concentration could be made, leaving open the possibility that some difference in the specific activities of alternatively spliced isoforms might have gone undetected. To address this issue directly, new 95-kD mouse agrin constructs (FIG. 1; C-Ag95_{z0/8}) were assembled in the pSecTag2 expression vector (Invitrogen), incorporating COOH-terminal myc and polyhistidine epitope tags, permitting purification and detection of the expressed protein and accurate concentration measurement to be made. (see Materials and Methods, above). Because the vast majority of agrin molecules expressed in brain include the 4 amino acid exon at the y site (Hoch et al., 1993; Li et al., 1997), all agrin constructs included the y4 exon, and only the properties of z-site variants were examined.

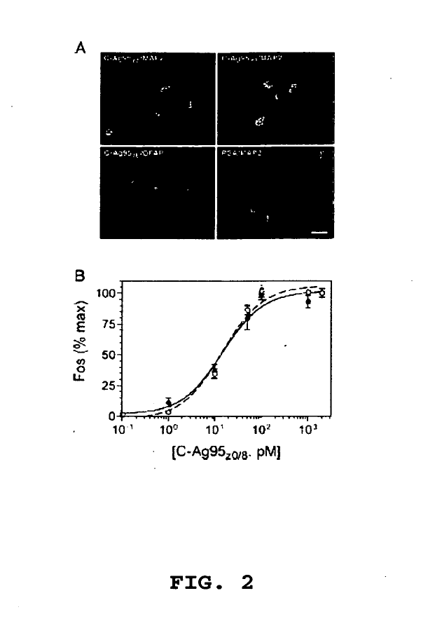

Rat rC-Agz0 and rC-Agz8 induce a neuron-specific increase in Fos expression (Hilgenberg et al., 1999). To confirm the properties of the corresponding mouse constructs, 12-d-old cortical cultures were treated with 1 nM purified mouse C-Ag95z0 or C-Ag95z8, and were then double labeled with antibodies against Fos and either microtubule-associated protein 2 (MAP2) or glial fibrillary acidic protein (GFAP) to identify neurons and glial cells, respectively. Consistent with our previous results, treatment with either C-Ag95z0 or C-Ag95z8 caused a marked increase in Fos expression in neurons, but not nonneuronal cells (FIG. 2). Although differences in the level of Fos expression between neurons were apparent, virtually all neurons (>90%) responded to the C-Ag95z0/8 treatment. In contrast, treatment with a similar concentration of prostate serum antigen control protein expressed in the same vector had no effect on Fos levels in either neurons or glia (FIG. 2). In light of these results, it appears that neither the myc epitope nor polyhistidine tags induce c-fos, nor do they affect the ability of the C-Ag95z0/8 sequences to do so.

Next, the specific activity of each z-site isoform was determined using an in situ enzyme-linked immunoassay (Hilgenberg et al., 1999) to examine the concentration dependence of c-fos induction by C-Ag95z0 and C-Ag95z8. As shown in FIG. 2B, both agrin isoforms induced c-fos in a concentration-dependent and saturable fashion. Fos expression curves were well fit by a single-site nonlinear regression model (R2≧0.94) predicting EC50 values of 11.93 ±0.44 pM for C-Ag95z0 and 12.67±0.58 pM for C-Ag95z8 (mean ±SEM). Similar EC50 values have also been reported for agrin induced clustering of AChR in muscle, but in contrast to the >1,000-fold difference in AChR clustering activity between isoforms (Ferns et al., 1992; Ruegg et al., 1992; Gesemann et al., 1995), the c-fos-inducing activity of C-Ag95z8 and C-Ag95z0 in neurons is the same.

### Example 2

### The 20-kD COOH-Terminal Portion of Agrin is Necessary and Sufficient for Signaling in Neurons

As a first step in localizing the structural domains responsible for signaling in neurons, we took advantage of knowledge gained from previous structural analyses of agrin function in muscle (Hoch et al., 1994; Gesemann et al., 1995, 1996) and divided C-Ag95_{z0/8} into two fragments. The first, an 75-kD NH 2-terminal fragment (FIG. 1; C-AgΔ20), which includes the 4 amino acid exon at the y site, has been shown to mediate agrin binding to α-dystroglycan (Gesemann et al., 1996; Hopf and Hoch, 1996). The remaining 20-kD COOH-terminal fragment contains the alternatively spliced z site (FIG. 1; C-Ag20_{z0} or C-Ag20_{z8}), and is homologous to the minimal agrin fragment able to induce clustering of AChR in cultured muscle cells (Gesemann et al., 1995).

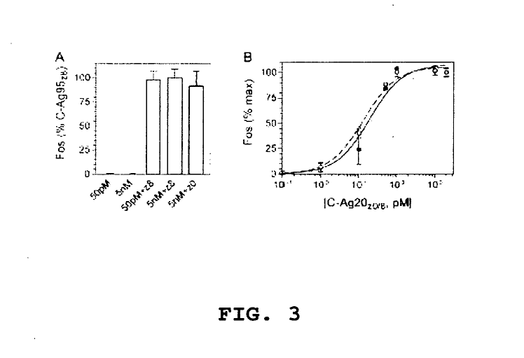

Treatment with C-AgΔ20, at a concentration equivalent to either a near saturating (50 pM) or supersaturating (5 nM) amount of C-Ag95_{z0/8}, had no effect on Fos expression in cortical cultures, suggesting that the active domain is not present within the C-AgΔ20 region (FIG. 3A). Agrin binding to α-dystroglycan has been shown to modulate agrin induced AChR clustering in muscle, and we considered the possibility of a similar function for α-dystroglycan signaling in neurons. However, coincubation with either an equal or 100-fold molar excess of C-AgΔ20 had no effect on Fos expression induced by either the C-Ag95_{z0} or C-Ag95_{z8} isoforms (FIG. 3A). Together, these data suggest that domains present within C-AgΔ20 are neither required for, nor modulate, agrin induction of c-fos in neurons.

The C-Ag20 fragments were both potent inducers of c-fos. As with C-Ag95_{z0/8}, c-fos induction by C-Ag20_{z0} (SEQ ID NO. 1) and C-Ag20_{z8} (SEQ ID NO. 2) was concentration-dependent and saturable (FIG. 3B). In fact, the EC₅₀ values obtained for the 20-kD fragments (C-Ag20_{z0}, 13.33±0.26 pM; C-Ag20_{z8}, 11.25 ±2.88 pM) were indistinguishable from each other and from those of the C-Ag95_{z0/8} isoforms. In light of these observations, we conclude that the structural domains that mediate agrin induction of c-fos are contained within the C-Ag20_{z0} fragment.

### Example 3

### Sequences Flanking the z Site are Critical for Agrin Signaling

Agrin's AChR clustering activity is regulated by alternative splicing at the z site. However, the observation that a peptide of the 8 amino acid alternatively spliced insert is itself inactive (Gesemann et al., 1995) suggests that domains important for agrin's bioactivity in muscle include not only the z site, but also amino acids that flank it. Because inclusion of alternatively spliced exons at the z site has no effect on agrin activity in neurons, we sought to determine the role of sequences surrounding the z site. To address this question, we deleted 37 amino acids from the NH₂ terminus of C-Ag20_{z0} to the border of the G3 domain, giving rise to a 15-kD COOH-terminal fragment, C-Ag15 (FIG. 1; SEQ. ID NO. 3).

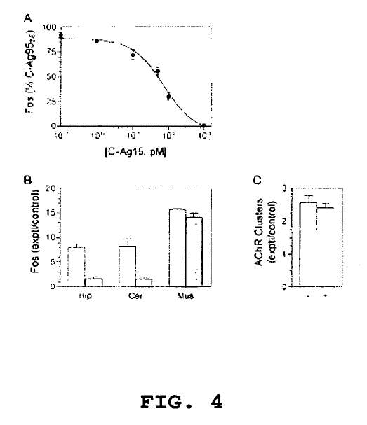

Treatment with C-Ag15 alone had no effect on the levels of Fos expression, even when present at a concentration fivefold above saturation for C-Ag20_{z0/8} (unpublished data). However, when added together with C-Ag95_{z8}, C-Ag15 appeared to inhibit the c-fos-inducing activity of the larger agrin fragment. To examine this effect in more detail, C-Ag15 dose-response studies were performed in the presence of a near saturating (50 pM) concentration of C-Ag95_{z8}. Increasing concentrations of C-Ag15 inhibited the c-fos-inducing activity of C-Ag95_{z8} (FIG. 4A). The curve was well fit by a nonlinear regression model for single-site competition (R²=0.95) predicting an IC₅₀ of 64.45±10.94 pM and close to a 1:1 agonist:antagonist molar ratio. Similar results were also obtained for competition against 50 pM C-Ag95_{z0} and the C-Ag20_{z0/8} isoforms (unpublished data).

To learn whether inhibition of agrin signaling by C-Ag15 might extend to other neurons or even muscle, mouse hippocampal and cerebellar neurons or chick skeletal myotubes were incubated with 50 pM C-Ag95_{z8} alone or in the presence of 1 nM C-Ag15, a concentration that blocks activity in cortical neurons. Treatment with C-Ag95_{z8} triggered a robust increase in Fos levels in both populations of neurons and in muscle compared with control sister cultures receiving vehicle (FIG. 4B). However, co-incubation with C-Ag15 inhibited Fos expression in the neurons, but had no effect on muscle. Because agrin's bioactivity in muscle is normally measured in terms of its AChR clustering activity, we also examined the ability of C-Ag15 to antagonize agrin-induced clustering of AChR. Chick myotubes were incubated overnight with 50 pM C-Ag95_{z8} alone or with 1 nM C-Ag15, AChR labeled with rhodamine-conjugated α-bungarotoxin, and the number of AChR clusters were determined. In line with the results of the Fos assay, C-Ag15 had no effect on the AChR clustering activity of C-Ag95_{z8} (FIG. 4C). Based on the results of these works, it appears that C-Ag15 is a specific antagonist for the neuronal receptor for agrin.

### Example 4

### C-Ag20z0/8 Rescues an Agrin-Deficient Neuronal Phenotype

Although induction of c-fos is a convenient reporter facilitating biochemical characterization of agrin signaling, its significance in terms of agrin function in CNS neurons is unknown. Recently, we have provided evidence that agrin plays an important role in neural differentiation by showing that agrin-deficient neurons exhibit reduced responses to glutamate both in cell culture and in vivo (Hilgenberg et al., 2002). Therefore, to learn more about the role of the 20-kD COOH-terminal region in agrin function, we tested the ability of the C-Ag20 isoforms to rescue the agrin deficiency.

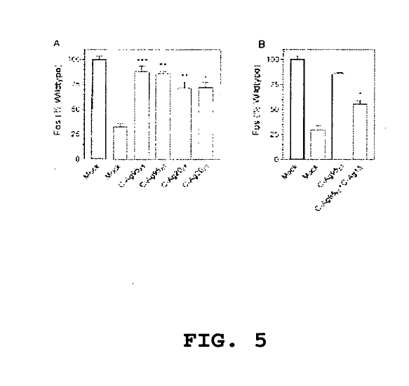

When grown in normal media, levels of glutamate-induced Fos expression in agrin-deficient neurons were reduced by ˜70% compared with wild type. However, consistent with our earlier findings, supplementing the growth media for 2 d with a saturating concentration of either C-Ag95_{z8} or C-Ag95_{z0} significantly increased the response of agrin-deficient neurons close to wild-type levels (FIG. 5A). Similar results were obtained when agrin-deficient neurons were grown in the presence of the C-Ag20_{z0/8} fragments. Compared with agrin-deficient neurons in vehicle-supplemented control media, treatment with either 5 nM C-Ag20_{z8} or C-Ag20_{z0} resulted in a significant 2.2-fold increase in glutamate response. Thus, not only do the C-Ag20_{z0/8} isoforms exhibit full activity in terms of their ability to induce c-fos, they are also competent to reverse the physiological deficit resulting from loss of expression of full length agrin in the agrin-deficient cultures.

Despite its apparent effectiveness as an antagonist of agrin-induced expression of c-fos, growth in media supplemented with up to 10 nM C-Ag15 had no effect on glutamate-induced expression of c-fos in either wild-type or heterozygous agrin-deficient neurons (unpublished data). This suggests that C-Ag15 is unable to block signaling by native agrin. However, the concentration of endogenous agrin in our cultures is unknown, and may exceed the maximal concentration of C-Ag15 we were able to use, especially if agrin is present as high density clusters on neuronal surface membranes (see below). As an alternative approach to examining the functional properties of the C-Ag15 fragment, we tested its ability to inhibit rescue of the agrin-deficient phenotype by C-Ag95_{z0/8}. Inclusion of 10 nM C-Ag15 in the growth medium significantly reduced the efficacy of 5 nM C-Ag95_{z0}, blocking slightly more than half (54%) of the rescue normally achieved by C-Ag95_{z0} alone (FIG. 5B). Similar results were also seen in single experiments using either C-Ag95_{z8} or C-Ag20_{z8} (unpublished data). Based on these observations we conclude that, under the appropriate conditions, C-Ag15 can inhibit agrin function in CNS neurons.

### Example 5

### Agrin Receptors are Concentrated at Neuron-Neuron Synapses

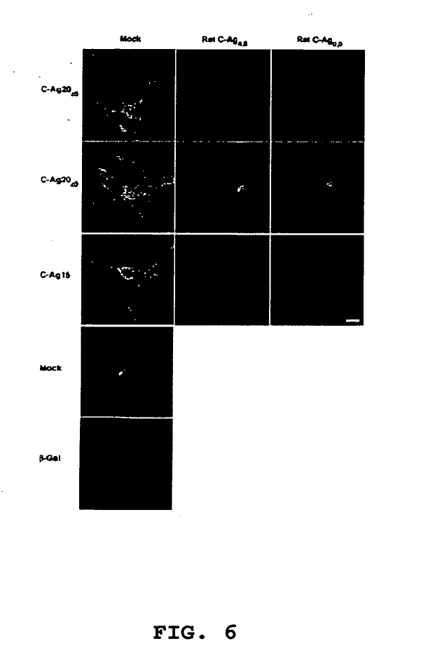

The works described so far provide evidence for a neuron specific agrin receptor, and identify a minimal fragment of agrin capable of activating it. Next, we sought to establish the cellular distribution of the neuronal receptor for agrin using the short agrin fragments as affinity ligands. Live cortical cultures were incubated in C-Ag20_{z8}, C-Ag20_{z0}, or C-Ag15, and were then labeled with an antibody against the polyhistidine epitope tag to visualize bound agrin. A similar pattern of labeling was evident for all three agrin fragments and appeared as bright puncta scattered over neuronal somata and neurites (FIG. 6). Labeling was specific in that nonneuronal cells were not labeled (unpublished data), and little or no labeling was evident when either the agrin fragments were omitted or a β-galactosidase-myc-6His vector control protein was used in their place (FIG. 6). More significantly, binding of all three agrin fragments was completely blocked in cultures labeled in the presence of a large excess of rC-Ag with both “neural” rC-Agy4_{z8} and “inactive” rC-Ag_{y0z0} appearing equally effective at blocking binding of either C-Ag20_{z} isoform or C-Ag15. Precise estimates of the concentration dependence of the competition between rC-Ag and the short mouse agrin fragments are difficult to obtain using immunofluorescence. However, NIH Image analysis of the mean pixel intensities obtained from fixed exposure photomicrographs of random fields revealed labeling to be reduced by ˜30% in cultures coincubated with a 1:1 molar ratio of C-Ag20_{z8} to rC-Ag_{y4z8}, and barely detectable at 1:100 (unpublished data). Together, these results provided strong evidence that the bioactivity of the different agrin isoforms shown herein is mediated through binding to a single population of agrin receptors expressed on cortical neuron cell membranes.

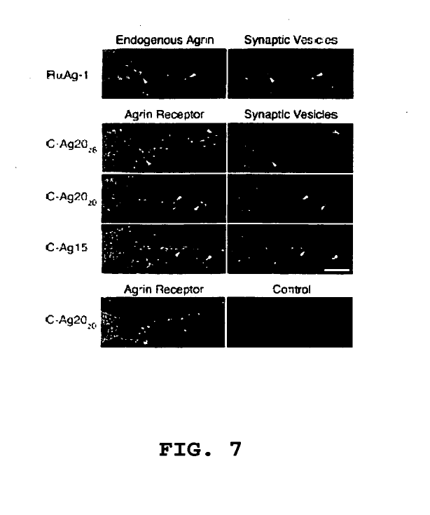

Previous papers have shown that agrin is present at neuron-neuron synapses in peripheral ganglia and retina (Mann and Kröger, 1996; Koulen et al., 1999; Gingras and Ferns, 2001). The punctate labeling observed with the short COOH-terminal agrin fragments suggested that receptors for agrin might also be synaptic. To test this hypothesis, we first determined the distribution of endogenous agrin on cultured cortical neurons by labeling live cultures with RαAg-1, a pan-specific anti-agrin serum, followed by fixation and labeling with a second antibody against the synaptic vesicle protein synaptophysin. Agrin immunostaining was distributed in patches on the neuronal cell bodies and neurites, and colocalized with synaptophysin-positive nerve terminals (FIG. 7). Although some variation in the intensity of the immunostaining was evident, few (if any) agrin-positive/synaptophysin-negative or synaptophysin-positive/agrin-negative patches were observed. Similar results were also obtained using an anti-agrin mAb (MAB5204; CHEMICON International, Inc.) and rabbit anti-synaptophysin antiserum (unpublished data). Therefore, we conclude that agrin is specifically localized at synapses formed between cultured cortical neurons.

Next, to learn whether agrin receptors also exhibit a synaptic pattern of expression, a parallel work was performed in which live cortical cultures were labeled with C-Ag20_{z8}, C-Ag20_{z0}, or C-Ag15 as before, followed by fixation and staining for synaptophysin. Irrespective of the agrin probe used, agrin receptor clusters exhibited a high degree of colocalization with the synaptic vesicle marker. Staining for synaptophysin was specific; normeuronal cells were not labeled by the synaptophysin antibody (unpublished data), and nerve terminal staining was not observed in cultures treated with C-Ag20_{z0} or other short fragments in the absence of the anti-synaptophysin antibody (FIG. 7). Together with the agrin immunostaining, the results of these studies show that both agrin and its receptor are concentrated at neuron-neuron synapses.

### Example 6

### Agrin Blocks Spontaneous Action Potentials in Cultured Cortical Neurons

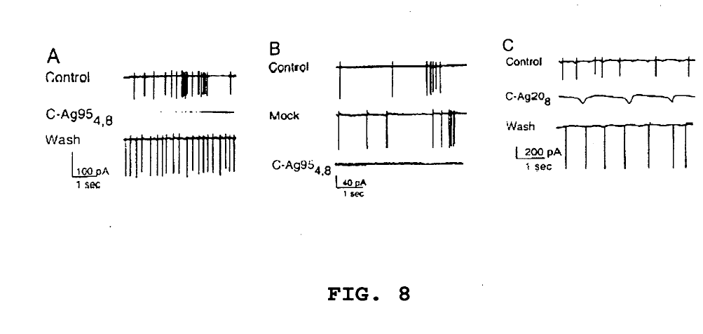

Electrophysiological studies employed extracellular recording to monitor the effect of agrin on the frequency of spontaneous action potentials in cultured cortical neurons. Spontaneous action potentials, at a frequency of 1-4 Hz, were recorded between 10-21 days in culture. Within 5 minutes of the addition of C-Ag95_{4,8}, to a concentration sufficient to saturate the c-fos induction assay, all spontaneous activity was blocked (FIG. 8). The effect of the agrin treatment was specific in that it was reversible and, therefore, not associated with a decline in the health of the cells or quality of the recording. Spike frequency was unaffected by treatment with conditioned medium from sham transfected cells normally used for expression of C-Ag95_{4,8}.

To learn whether agrin induced changes in spike frequency were associated with activation of the neuronal receptor for agrin, we tested the effects of C-Ag208. Treatment with C-Ag20₈ reversibly blocked spontaneous action potentials (FIG. 8C). Thus, a single signal pathway appears to be responsible for agrin regulation of neuronal excitability, rescue of an agrin-deficient phenotype, and induction of c-fos. Agrin binds dystroglycan and several other cell surface molecules. The absence of binding sites for these putative agrin receptors in C-Ag20₈ rules out a role for these molecules in agrin dependent modulation of neuronal excitability.

### Example 7

### Agrin Increases Activity at Inhibitory Synapses

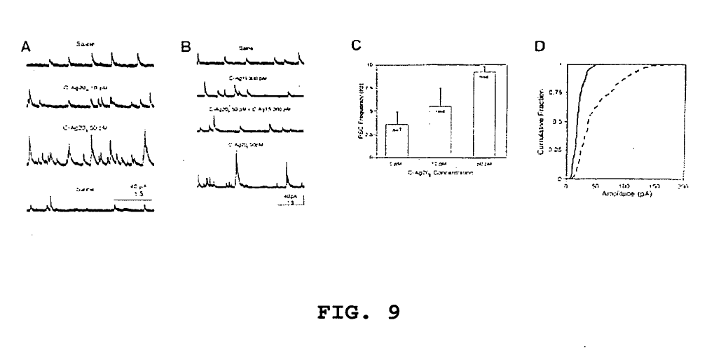

A possible mechanism whereby agrin might exert its effects was suggested by a record obtained from a neuron where a population of small, slow, events was evident in addition to action potentials (FIG. 8C). In this cell, suppression of spontaneous action potentials by treatment with C-Ag20₈ was accompanied by an increase in amplitude of the slow events, consistent with the possibility agrin might increase the level of inhibition within the network. To examine this hypothesis, whole cell voltage clamp was used to study the effects of agrin on spontaneous inhibitory postsynaptic currents (sIPSCs; FIG. 9). Consistent with the inhibition of action potentials, C-Ag20₈ produced a reversible increase in amplitude and frequency of sIPSCs. The reaction to agrin was concentration dependent in that both the number of neurons responding and frequency of sIPSCs increased with the concentration of C-Ag20₈ used. As predicted for a single agrin signal pathway, C-Ag15 proved to be an effective antagonist to the larger fragment (FIG. 9B).

Several papers have provided evidence that agrin plays a role in differentiation of CNS neurons. In particular, using the immediate early gene c-fos as a reporter, we have characterized an agrin-signaling pathway that influences development of neuronal responses to excitatory neurotransmitters and depolarization (Hilgenberg et al., 1999, 2002). To learn more about the receptor mediating agrin's function in brain, we have analyzed the structural features of agrin required for activation of its signaling pathway. Our results confirm agrin's activity in CNS neurons is independent of alternative splicing at the z site, and demonstrate further that agrin's functional domains reside within 20 kD of its COOH terminus. Also, we show that the receptor mediating these agrin-dependent signals is colocalized with agrin at neuron-neuron synapses.

Initial attempts to characterize molecules that might mediate agrin-induced clustering of AChR identified α-dystroglycan as a major agrin-binding protein in muscle cell membranes (Campanelli et al., 1994; Gee et al., 1994). Although it now seems unlikely to represent the “functional” agrin receptor, evidence suggests that α-dystroglycan still plays an important role in consolidation and maintenance of AChR clusters once formed (Jacobson et al., 1998, 2001; Heathcote et al., 2000). Dystroglycan is also found in mammalian brain, where its expression and synaptic localization have suggested a role in synaptic function (Tian et al., 1996; Zaccaria et al., 2001; Lévi et al., 2002). Interestingly, several biochemical properties of agrin induction of c-fos in CNS neurons, including Ca²⁺ dependence, inhibition by heparin, and ability to bind z⁺ and z⁻ isoforms (Hilgenberg et al., 1999), are consistent with agrin binding to α-dystroglycan (Sugiyama et al., 1994; Gesemann et al., 1996). High affinity binding of agrin to α-dystroglycan is mediated by a region that includes the first two laminin G domains (Gesemann et al., 1996; Hopf and Hoch, 1996). However, C-AgΔ20, which contains the G1 and G2 domains, neither induced c-fos nor inhibited the activity of the larger fragments. Conversely, both C-Ag20_{z0/8} isoforms, lacking domains for high affinity binding to α-dystroglycan, induced c-fos and rescued the agrin-deficient phenotype with a similar efficacy as their 95-kD C-Ag_{z0/8} counterparts. Although these results do not rule out a role for α-dystroglycan mediating other aspects of agrin signaling, α-dystroglycan is unlikely to be a component of the agrin receptor mediating the responses measured here.

Also, we considered the possibility that the MuSK-MASC complex might mediate agrin signaling in neurons. Both neural and muscle signaling pathways are activated by picomolar concentrations of agrin, are inhibited by heparin, and are Ca²⁺ dependent (Hilgenberg et al., 1999). Even more striking is the fact that the same minimal agrin fragment is sufficient to activate the MuSK-MASC complex (Gesemann et al., 1995) and neuronal receptor; evidence that significant structural homology exists between the two receptors. However, despite these similarities, we believe the neuronal and muscle receptors are distinct. MuSK expression has not been detected in either embryonic or adult mammalian brain (Valenzuela et al., 1995). Moreover, although a critical determinant of agrin's AChR clustering activity in muscle (Ferns et al., 1992; Ruegg et al., 1992; Gesemann et al., 1995), alternative splicing at the z site has no effect on agrin's bioactivity assayed here, even in the case of the smallest active fragment where variations in structure might be expected to exert the greatest effect. The finding that the C-Ag20_{z0/8} fragments are fully active also contrasts with observations in muscle where the AChR clustering activity of the corresponding fragment is >100-fold lower than the 95-kD C-Ag polypeptide from which it was derived (Gesemann et al., 1995). Together with the inability of C-Ag15 to antagonize agrin induction of either c-fos or AChR clustering in muscle, these data point to a fundamental difference in the receptors that catalyze the agrin response in nerve and muscle cells.

The inhibition of c-fos induction by C-Ag15, its ability to block rescue of the agrin-deficient phenotype, distribution of binding sites, and ability of different agrin isoforms to block binding to them argue that C-Ag15 and the active agrin polypeptide fragments compete for a common binding site on the neuronal receptor for agrin. How then might C-Ag15 bind to the receptor but not activate it? One possibility is that deletion of the 5-kD NH₂-terminal region may have disordered secondary or tertiary structures in the remaining G3 and COOH-terminal domain necessary for signaling. However, that such a structural change could occur without affecting the apparent affinity of C-Ag15 binding to the receptor seems improbable. Alternatively, activation of the receptor may require agrin binding to two sites, with the 5-kD NH₂-terminal region of C-Ag20_{z0/8} targeted to the second site. The agrin receptor in muscle is made up of two components (MuSK and MASC) although agrin appears only to bind directly to MASC (Glass et al., 1996). Whereas a similar two-component model of the neuronal receptor for agrin would be consistent with our data, the fact that C-Ag15 has no effect on agrin's bioactivity in muscle suggests that, although functionally homologous to MASC, the agrin-binding component of the neuronal receptor would be structurally distinct.

Blocking agrin expression with antisense oligonucleotides inhibits synapse formation and alters synaptic function in CNS neurons (Ferreira, 1999; Böse et al., 2000). The ability of rC-Ag95_{z8} (but not rC-Ag95_{z0}) to rescue changes induced by the antisense oligonucleotides suggests these effects are mediated by a loss of isoform-specific signals associated with agrin's COOH-terminal domains (Ji et al., 1998; Böse et al., 2000). These observations contrast to our own findings that, in terms of their ability to induce c-fos and rescue an agrin mutant phenotype, the bioactivity of even the shortest alternatively spliced active fragments are the same. One possible explanation for this apparent inconsistency is that different domains within the 95-kD COOH-terminal region of agrin might mediate distinct responses through specific receptors. In this regard, it is interesting to note that a recent paper showed that in addition to α-dystroglycan/heparin binding, the 95-kD COOH-terminal region of agrin contains three other sites that interact with neuronal cell surface receptors (Burgess et al., 2002). Two of these sites are targeted to integrins, one of which modulates agrin's AChR clustering activity, whereas the receptor for the third site, located within 20 kD of agrin's COOH terminus, has yet to be identified. It will be interesting, in light of these observations, to determine whether C-Ag15 inhibition is limited to the bioassays used here, or whether it extends to other agrin-dependent neuronal responses.

Agrin induces expression of c-fos in CNS neurons, and mutation of the agrin gene decreases neuronal responses to excitatory neurotransmitters (Hilgenberg et al., 2002). Although neurons express multiple molecules capable of binding agrin, several lines of evidence support the conclusion that the agrin-binding sites visualized using the affinity probes are the receptors responsible for the physiologic responses to agrin reported here and earlier (Hilgenberg et al., 1999). First, the apparent affinities and potency of the various agrin fragments, in terms of their c-fos-inducing activity or ability to rescue the agrin-deficient phenotype, are the same. Moreover, the ability of the smallest agrin fragment, C-Ag15, to inhibit both c-fos induction and rescue of the agrin-deficient phenotype is strong evidence for a common receptor mediating both activities. A similar profile was also apparent for the binding properties of the agrin fragments when used to visualize the distribution of the agrin receptors. Consistent with the biochemical studies, neurons were clearly labeled using agrin concentrations in the picomolar range irrespective of z site composition. Most importantly, the distribution of high density binding sites for C-Ag15 and their sensitivity to blockade by rC-Ag_{z0/8} was indistinguishable from that observed with either C-Ag20_{z0/8} constructs.

Previous papers have shown that agrin is concentrated at cholinergic synapses formed between cultured sympathetic neurons (Gingras and Ferns, 2001) and GABAergic synapses in the retina (Mann and Kröger, 1996; Koulen et al., 1999). Cultured cortical neurons receive both glutamatergic and GABAergic inputs (Li et al., 1997), and because no evidence of synaptic contacts lacking agrin was found, we can extend these findings to include glutamatergic synapses as well. A similarly high correlation between agrin receptors and nerve terminals was also evident, suggesting that agrin and its receptor colocalize at synaptic sites. Together, these observations suggest that regardless of neurotransmitter phenotype, synapses are likely to be significant sites of agrin action. Consistent with these conclusions, agrin has been implicated in regulating a number of pre- and postsynaptic properties of cholinergic, glutamatergic, and GABAergic neurons (Ferreira, 1999; Bose et al., 2000; Gingras et al., 2002). Higher resolution techniques than those used here will be required to determine the agrin receptor's subcellular distribution. However, if all agrin receptors are presynaptic, then a long-range retrograde signal would be needed to account for agrin's ability to rapidly induce c-fos. Therefore, it seems likely that at least some agrin receptors, for example, those at or near the neuronal soma, are postsynaptic.

At the neuromuscular junction, agrin is anchored to the synaptic basal lamina by a laminin-binding domain present within its NH₂ terminus (Denzer et al., 1995). However, the mechanism controlling agrin's spatial organization in neurons, and in particular, its concentration at synaptic sites, is unknown. Neuron-neuron synapses lack a basal lamina; moreover, the short NH₂-terminal form of agrin (SN-agrin) expressed in CNS neurons not only lacks the laminin-binding domain, but consistent with a type II membrane protein, is oriented such that its NH₂ terminus is cytoplasmic (Burgess et al., 2000; Neumann et al., 2001). That signals embedded within this cytoplasmic domain might play a role in agrin's subcellular localization also seems unlikely. Fusion of the SN-agrin NH₂ terminus to YFP directs YFP to neuronal membranes, but the pattern of expression is uniform rather than concentrated at synaptic sites (Burgess et al., 2002). In light of these findings, agrin's spatial distribution would seem to be defined by interactions between its extracellular domains and candidate neuronal cell surface agrin-binding proteins such as neural cell adhesion molecules, integrins, and α-dystroglycan. Clearly, the high affinity and synapse-specific pattern of expression of the neuronal receptor for agrin are also consistent with the possibility that, in addition to signal transduction, it could play a role sculpting agrin's spatial distribution. Future studies comparing the time course of agrin and agrin receptor expression and any effect C-Ag15 might have on agrin's distribution will be helpful in testing this hypothesis.

### REFERENCES

Böse, C. M., D. Qiu, A. Bergamaschi, B. Gravante, M. Bossi, A. Villa, F. Rupp, and A. Malgaroli. 2000. Agrin Controls Synaptic Differentiation In Hippocampal. Neurons. J. Neurosci. 20: 9086-9095.
Burgess, R. W., W. C. Skarnes, and J. R. Sanes. 2000. Agrin Isoforms With Distinct Amino Termini: Differential Expression, Localization, And Function. J. Cell Biol. 151: 41-52.
Burgess, R. W., D. K. Dickman, L. Nunez, D. J. Glass, and J. R. Sanes. 2002. Mapping Sites Responsible For Interactions Of Agrin With Neurons. J. Neurochem. 83: 271-284.
Campagna, J. A., M. Ruegg, and J. L. Bixby. 1995. Agrin Is A Differentiation-Inducing “Stop Signal” For Motorneurons In Vitro. Neuron. 15: 1365-1374.
Campanelli, J. T., S. L. Roberds, K. P. Campbell, and R. H. Scheller. 1994. A Role For Dystrophin-Associated Glycoproteins And Utrophin In Agrin-Induced AChR Clustering. Cell. 77: 663-674.
Denzer, A. J., M. Gesemann, B. Schumacher, and M. A. Ruegg. 1995. An Aminoterminal Extension Is Required For The Secretion Of Chick Agrin And Its Binding To Extracellular Matrix. J. Cell Biol. 131: 1547-1560.
Ferns, M., W. Hoch, J. T. Campanelli, F. Rupp, Z. W. Hall, and R. H. Scheller. 1992. RNA Splicing Regulates Agrin-Mediated Acetylcholine Receptor Clustering Activity On Cultured Myotubes. Neuron. 8: 1079-1086.
Ferns, M. J., J. T. Campanelli, W. Hoch, R. H. Scheller, and Z. Hall. 1993. The Ability Of Agrin To Cluster Achrs Depends On Alternative Splicing And On Cell Surface Proteoglycans. Neuron. 11: 491-502.
Ferreira, A. 1999. Abnormal Synapse Formation In Agrin-Depleted Hippocampal Neurons. J. Cell Sci. 112: 4729-4738.
Gautam, M., P. G. Noakes, L. Moscoso, F. Rupp, R. H. Scheller, J. P. Merlie, and J. R. Sanes. 1996. Defective Neuromuscular Synaptogenesis In Agrin-Deficient Mutant Mice. Cell. 85: 525-536.
Gee, S. H., F. Montanaro, M. H. Lindenbaum, and S. Carbonetto. 1994. Dystroglycan-α, A Dystrophin-Associated Glycoprotein, Is A Functional Agrin Receptor. Cell. 77: 675-686.
Gesemann, M., A. J. Denzer, and M. A. Ruegg. 1995. Acetylcholine Receptor-Aggregating Activity Of Agrin Isoforms And Mapping Of The Active Site. J. Cell Biol. 128: 625-636.
Gesemann, M., V. Cavalli, A. J. Denzer, A. Brancaccio, B. Schumacher, and M. A. Ruegg. 1996. Alternative Splicing Of Agrin Alters Its Binding To Heparin, Dystroglycan, And The Putative Agrin Receptor. Neuron. 16: 755-767.
Gingras, J., and M. Ferns. 2001. Expression And Localization Of Agrin During Sympathetic Synapse Formation In Vitro. J. Neurobiol. 48: 228-242.
Gingras, J., S. Rassadi, E. Cooper, and M. Ferns. 2002. Agrin Plays An Organizing Role In The Formation Of Sympathetic Synapses. J. Cell Biol. 158: 1109-1118.
Glass, D. J., D. C. Bowen, T. N. Stitt, C. Radziejewski, J. Bruno, T. E. Ryan, D. R. Gies, S. Shah, K. Mattson, S. J. Burden, et al. 1996. Agrin Acts Via a MuSK Receptor Complex. Cell. 85: 513-524.
Heathcote, R. D., J. M. Ekman, K. P. Campbell, and E. W. Godfrey. 2000. Dystroglycan Overexpression In Vivo Alters Acetylcholine Receptor Aggregation At The Neuromuscular Junction. Dev. Biol. 227: 595-605.
Hilgenberg, L. G. W., K. D. Ho, D. Lee, D. O'Dowd, and M. A. Smith. 2002. Agrin Regulates Neuronal Responses To Excitatory Neurotransmitters In Vitro And In Vivo. Mol. Cell. Neurosci. 19: 97-110.
Hilgenberg, L. G. W., C. L. Hoover, and M. A. Smith. 1999. Evidence Of An Agrin Receptor In Cortical Neurons. J. Neurosci. 19: 7384-7393.
Hoch, W. 1999. Formation Of The Neuromuscular Junction. Agrin And Its Unusual Receptors. Eur. J. Biochem. 265: 1-10.
Hoch, W., M. Ferns, J. T. Campanelli, Z. W. Hall, and R. H. Scheller. 1993. Developmental Regulation Of Highly Active Alternatively Spliced Forms Of Agrin. Neuron. 11: 479-490.
Hoch, W., J. T. Campanelli, S. Harrison, and R. H. Scheller. 1994. Structural Domains Of Agrin Required For Clustering Of Nicotinic Acetylcholine Receptors. EMBO J. 13: 2814-2821.
Hopf, C., and W. Hoch. 1996. Agrin Binding to α-dystroglycan. J. Biol. Chem. 271: 5231-5236.
Jacobson, C., F. Montanaro, M. Lindenbaum, S. Carbonetto, and M. Ferns. 1998. α-Dystroglycan Functions In Acetylcholine Receptor Aggregation But Is Not A Coreceptor For Agrin-MuSK Signaling. J. Neurosci. 18: 6340-6348.
Jacobson, C., P. D. Cote, S. G. Rossi, R. L. Rotundo, and S. Carbonetto. 2001. The Dystroglycan Complex Is Necessary For Stabilization Of Acetylcholine Receptor Clusters At Neuromuscular Junctions And Formation Of The Synaptic Basement Membrane. J. Cell Biol. 152: 435-450.
Ji, R.-R., C. M. Böse, C. Lesuisse, D. Qiu, J. C. Huang, Q. Zhang, and F. Rupp. 1998. Specific Agrin Isoforms Induce cAMP Response Element Binding Protein Phosphorylation In Hippocampal Neurons. J. Neurosci. 18: 9695-9702.
Koulen, P., L. S. Honig, E. L. Fletcher, and S. Kroger. 1999. Expression, Distribution And Ultrastructural Localization Of The Synapse-Organizing Molecule Agrin In The Mature Avian Retina. Eur. J. Neurosci. 11: 4188-4196.
Lévi, S., R. M. Grady, M. D. Henry, K. P. Campbell, J. R. Sanes, and A. M. Craig. 2002. Dystroglycan Is Selectively Associated With Inhibitory GABAergic Synapses But Is Dispensable For Their Differentiation. J. Neurosci. 22: 4274-4285.
Li, Z., J. L. Massengill, D. K. O'Dowd, and M. A. Smith. 1997. Agrin Gene Expression In Mouse Somatosensory Cortical Neurons During Development In Vivo And In Cell Culture. Neuroscience. 79: 191-201.
Li, Z., L. G. W. Hilgenberg, D. K. O'Dowd, and M. A. Smith. 1999. Formation Of Functional Synaptic Connections Between Cultured Cortical Neurons From Agrin-Deficient Mice. J. Neurobiol. 39: 547-557.
Mann, S., and S. Kröger. 1996. Agrin Is Synthesized By Retinal Cells And Colocalizes With Gephyrin. Mol. Cell. Neurosci. 8: 1-13.
Mantych, K. B., and A. Ferreira. 2001. Agrin Differentially Regulates The Rates Of Axonal And Dendritic Elongation In Cultured Hippocampal Neurons. J. Neurosci. 21: 6802-6809.
Neumann, F. R., G. Bittcher, M. Annies, B. Shumacher, S. Kröger, and M. A. Ruegg. 2001. An Alternative Amino-Terminus Expressed In The Central Nervous System Converts Agrin To A Type II Transmembrane Protein. Mol. Cell. Neurosci. 17: 208-225.
Ruegg, M. A., K. W. K. Tsim, S. E. Horton, S. Kröger, and U. J. McMahan. 1992. The Agrin Gene Codes For A Family Of Basal Lamina Proteins That Differ In Function And Distribution. Neuron. 8: 691-699.
Rupp, F., T. H. Özçelik, M. Linial, K. Peterson, U. Francke, and R. Scheller. 1992. Structure and Chromosomal Localization Of The Mammalian Agrin Gene. J. Neurosci. 12: 3535-3544.
Sugiyama, J., D. C. Bowen, and Z. W. Hall. 1994. Dystroglycan Binds Nerve And Muscle Agrin. Neuron. 13: 103-115.
Tian, M., C. Jacobson, S. H. Gee, K. P. Campbell, S. Carbonetto, and M. Jucker. 1996. Dystroglycan In The Cerebellum Is A Laminin Alpha 2-Chain Binding Protein At The Glial-Vascular Interface And Is Expressed In Purkinje Cells. Eur. J. Neurosci. 8: 2739-2747.
Valenzuela, D. M., T. N. Stitt, P. S. DiStefano, E. Rojas, K. Mattsson, D. L. Compton, L. Nunez, J. S. Park, J. L. Stark, D. R. Gies, et al. 1995. Receptor Tyrosine Kinase Specific For The Skeletal Muscle Lineage: Expression In Embryonic Muscle, At The Neuromuscular Junction, And After Injury. Neuron. 15: 573-584.
Zaccaria, M. L., F. Di Tommaso, A. Brancaccio, P. Paggi, and T. C. Petrucci. 2001. Dystroglycan Distribution In Adult Mouse Brain: A Light And Electron Microscopy Study. Neuroscience. 104: 311-324.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

1. A method for controlling seizures in patients diagnosed with epilepsy, comprising administering to an individual diagnosed with epilepsy a therapeutically effective amount of a polypeptide comprising an approximately 15-kD C-terminal agrin fragment.

2. The method of claim 1, wherein the polypeptide has the amino acid sequence identified as SEQ. ID NO. 3.

3. The method of claim 1, wherein the polypeptide is a homolog of the polypeptide having the amino acid sequence identified as SEQ. ID NO. 3.

4. The method of claim 1, wherein the polypeptide is a derivative of the polypeptide having the amino acid sequence identified as SEQ. ID NO. 3.

5. The method of claim 1, wherein the polypeptide is a peptidomimetic of the polypeptide having the amino acid sequence identified as SEQ. ID NO. 3.

6. The method of claim 1, wherein the individual is a human.

7. The method of claim 1, wherein the polypeptide is administered by regional perfusion to the central nervous system.

8. The method of claim 1, wherein the polypeptide is administered by intraperitoneal injection.

9. A method for treating traumatic injury to the central nervous system, comprising administering to an individual diagnosed with traumatic injury a therapeutically effective amount of a polypeptide comprising an approximately 15-kD C-terminal agrin fragment.

10. The method of claim 9, wherein the polypeptide has the amino acid sequence identified as SEQ. ID NO. 3.

11. The method of claim 9, wherein the polypeptide is a homolog of the polypeptide having the amino acid sequence identified as SEQ. ID NO. 3.

12. The method of claim 9, wherein the polypeptide is a derivative of the polypeptide having the amino acid sequence identified as SEQ. ID NO. 3.

13. The method of claim 9, wherein the polypeptide is a peptidomimetic of the polypeptide having the amino acid sequence identified as SEQ. ID NO. 3.

14. The method of claim 9, wherein the individual is a human.

15. A method for rescuing an agrin deficient phenotype in an individual in need thereof, comprising administering to the individual a therapeutically effective amount of a polypeptide comprising an approximately 20-kD C-terminal agrin fragment.

16. The method of claim 15, wherein the polypeptide has the amino acid sequence identified as SEQ. ID NO. 1.

17. The method of claim 15, wherein the polypeptide has the amino acid sequence identified as SEQ. ID NO. 2.

18. The method of claim 15, wherein the polypeptide is a homolog of the polypeptide having the amino acid sequence identified as SEQ. ID NO. 1.

19. The method of claim 15, wherein the polypeptide is a derivative of the polypeptide having the amino acid sequence identified as SEQ. ID NO. 1.

20. The method of claim 15, wherein the polypeptide is a homolog of the polypeptide having the amino acid sequence identified as SEQ. ID NO. 2.

21. The method of claim 15, wherein the polypeptide is a derivative of the polypeptide having the amino acid sequence identified as SEQ. ID NO. 2.

22. The method of claim 15, wherein the polypeptide is a peptidomimetic of the polypeptide having the amino acid sequence identified as SEQ. ID NO. 1.

23. The method of claim 15, wherein the polypeptide is a peptidomimetic of the polypeptide having the amino acid sequence identified as SEQ. ID NO. 2.

24. The method of claim 15, wherein the individual is a human.

25. The method of claim 15, wherein the polypeptide is administered by regional perfusion to the central nervous system.

26. The method of claim 15, wherein the polypeptide is administered by intraperitoneal injection.

27. A method of manufacturing a medicament for use in treating seizures in a mammal, the method comprising:
(a) providing a composition in dosage form, which comprises a synthetic polypeptide comprising an approximately 15-kD C-terminal agrin fragment;
(b) packaging the composition; and
(c) providing the package with a label instructing a user to administer the composition as a medicament for use in treating seizures in a mammal.

28. The method of claim 27, wherein the polypeptide has the amino acid sequence identified as SEQ. ID NO. 3.

29. The method of claim 27, wherein the polypeptide is a homolog of the polypeptide having the amino acid sequence identified as SEQ. ID NO. 3.

30. The method of claim 27, wherein the polypeptide is a derivative of the polypeptide having the amino acid sequence identified as SEQ. ID NO. 3.

31. The method of claim 27, wherein the polypeptide is a peptidomimetic of the polypeptide having the amino acid sequence identified as SEQ. ID NO. 3.

32. The method of claim 27, wherein the mammal is a human.

33. A method of manufacturing a medicament for use in rescuing an agrin-deficient phenotype in a mammal, the method comprising:
(a) providing a composition in dosage form, which comprises a synthetic polypeptide comprising an approximately 20-kD C-terminal agrin fragment;
(b) packaging the composition; and
(c) providing the package with a label instructing a user to administer the composition as a medicament for use in rescuing an agrin-deficient phenotype in a mammal.

34. The method of claim 33, wherein the polypeptide has the amino acid sequence identified as SEQ. ID NO. 1.

35. The method of claim 33, wherein the polypeptide has the amino acid sequence identified as SEQ. ID NO. 2.

36. The method of claim 33, wherein the polypeptide is a homolog of the polypeptide having the amino acid sequence identified as SEQ. ID NO. 1.

37. The method of claim 33, wherein the polypeptide is a derivative of the polypeptide having the amino acid sequence identified as SEQ. ID NO. 1.

38. The method of claim 33, wherein the polypeptide is a homolog of the polypeptide having the amino acid sequence identified as SEQ. ID NO. 2.

39. The method of claim 33, wherein the polypeptide is a derivative of the polypeptide having the amino acid sequence identified as SEQ. ID NO. 2.

40. The method of claim 33, wherein the polypeptide is a peptidomimetic of the polypeptide having the amino acid sequence identified as SEQ. ID NO. 1.

41. The method of claim 33, wherein the polypeptide is a peptidomimetic of the polypeptide having the amino acid sequence identified as SEQ. ID NO. 2.

42. The method of claim 33, wherein the mammal is a human.

43. A purified polypeptide, the amino acid sequence of which consists of SEQ ID NO. 1.

44. A purified polypeptide, the amino acid sequence of which consists of SEQ ID NO. 2.

45. A purified polypeptide, the amino acid sequence of which consists of SEQ ID NO. 3.

46. A purified polypeptide, the amino acid sequence of which comprises a sequence at least 70% identical to SED ID NO. 1.

47. A purified polypeptide, the amino acid sequence of which comprises a sequence at least 70% identical to SED ID NO. 2.

48. A purified polypeptide, the amino acid sequence of which comprises a sequence at least 70% identical to SED ID NO. 3.

49. A purified polypeptide, the amino acid sequence of which comprises SEQ ID NO. 1, or SEQ ID NO. 1 with at least one conservative amino acid substitution.

50. A purified polypeptide, the amino acid sequence of which comprises SEQ ID NO. 2, or SEQ ID NO. 2 with at least one conservative amino acid substitution.

51. A purified polypeptide, the amino acid sequence of which comprises SEQ ID NO. 3, or SEQ ID NO. 3 with at least one conservative amino acid substitution.
